(19) United States
(12) Patent Application Publication
(10) Pub. No.: US 2009/0149878 A1
(43) Pub. Date: Jun. 11, 2009

(54) BONE TREATMENT SYSTEMS AND METHODS

(76) Inventors: Csaba Truckai, Saratoga, CA (US); Robert Luzzi, Pleasanton, CA (US); John H. Shadduck, Tiburon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/952,942

(22) Filed: Dec. 7, 2007

(51) Int. Cl. A61B 17/34; A61B 17/58

(52) U.S. Cl. 606/186; 606/92

(57) ABSTRACT

The present invention relates in certain embodiments to systems for treating vertebral compression fractures. In one embodiment, a trocar with a flexible tip is provided to create a curved path in cancellous bone. An injector can be introduced into the vertebra in communication with the curved path for delivery of bone fill material into the curved path. Optionally, thermal energy can be applied to the bone fill material prior to injection into the curved path in cancellous bone to alter a property (e.g., viscosity) of the bone fill material.

### CROSS-REFERENCE TO RELATED APPLICATIONS

This application claims the benefit of U.S. Provisional Patent Application No. 60/837,592 filed Dec. 7, 2006, the entire contents of which are incorporated herein by reference and should be considered a part of this specification. This application is also related to the following U.S. Patent Applications: application Ser. No. 11/469,764 filed Sep. 1, 2006; application Ser. No. 11/165,652 filed Jun. 24, 2005; App. No. 60/726,152 filed Oct. 13, 2005 titled Bone Treatment Systems and Methods; and application Ser. No. 11/209,035 filed Aug. 22, 2005. The entire contents of all of the above applications are hereby incorporated by reference and should be considered a part of this specification.

### BACKGROUND OF THE INVENTION

1. Field of the Invention

The present invention relates in certain embodiments to systems for treating vertebral compression fractures. In one embodiment, systems and methods are provided for creating a curved path in bone in a desired plane and for introducing a bone fill material into said curved path. In another embodiment, energy can be applied to the bone fill material flow to alter a property (e.g., viscosity) of the bone fill material. In still another embodiment, the system can include a tubular sleeve that provides a port that can engage a cortical bone portion of the bone to allow instrument exchange therethrough.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the population affected will grow steadily as life expectancy increases. Osteoporosis affects the entire skeleton but most commonly causes fractures in the spine and hip. Spinal or vertebral fractures also cause other serious side effects, with patients suffering from loss of height, deformity and persistent pain which can significantly impair mobility and quality of life. Fracture pain usually lasts 4 to 6 weeks, with intense pain at the fracture site. Chronic pain often occurs when one vertebral level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporosis bone, the sponge-like cancellous bone has pores or voids that increase in dimension making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. Vertebroplasty is the percutaneous injection of PMMA (polymethylmethacrylate) into a fractured vertebral body via a trocar and cannula. The targeted vertebrae are identified under fluoroscopy. A needle is introduced into the vertebrae body under fluoroscopic control, to allow direct visualization. A bilateral transpedicular (through the pedicle of the vertebrae) approach is typical but the procedure can be done unilaterally. The bilateral transpedicular approach allows for more uniform PMMA infill of the vertebra.

In a bilateral approach, approximately 1 to 4 ml of PMMA is used on each side of the vertebra. Since the PMMA needs to be forced into the cancellous bone, the techniques require high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasation are critical to the technique-and the physician terminates PMMA injection when leakage is evident. The cement is injected using syringes to allow the physician manual control of injection pressure.

Kyphoplasty is a modification of percutaneous vertebroplasty. Kyphoplasty involves a preliminary step consisting of the percutaneous placement of an inflatable balloon tamp in the vertebral body. Inflation of the balloon creates a cavity in the bone prior to cement injection. The proponents of percutaneous kyphoplasty have suggested that high pressure balloon-tamp inflation can at least partially restore vertebral body height. In kyphoplasty, some physicians state that PMMA can be injected at a lower pressure into the collapsed vertebra since a cavity exists, when compared to conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles.

Leakage of PMMA during vertebroplasty can result in very serious complications including compression of adjacent structures that necessitate emergency decompressive surgery. See “Anatomical and Pathological Considerations in Percutaneous Vertebroplasty and Kyphoplasty: A Reappraisal of the Vertebral Venous System”, Groen, R. et al, Spine Vol. 29, No. 13, pp 1465-1471 2004. Leakage or extravasation of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (5/2004) pp. 478-82, (http://www.jkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2):175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al, “Asymptomatic diffuse pulmonary embolism caused by acrylic cement: an unusual complication of percutaneous vertebroplasty”, Ann. Rheum. Dis. 2003; 62:85-86. The vapors from PMMA preparation and injection also are cause for concern. See Kirby, B, et al., “Acute bronchospasm due to exposure to polymethylmethacrylate vapors during percutaneous vertebroplasty”, Am. J. Roentgenol. 2003; 180:543-544.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon applies also compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which crushes and compacts cancellous bone. Expansion of the balloon under high pressures close to cortical bone can fracture the cortical bone, typically the endplates, which can cause regional damage to the cortical bone with the risk of cortical bone necrosis. Such cortical bone damage is highly undesirable as the endplate and adjacent structures provide nutrients for the disc.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a general need to provide bone cements and methods for use in treatment of vertebral compression fractures that provide a greater degree of control over introduction of cement and that provide better outcomes. The present invention meets this need and provides several other advantages in a novel and nonobvious manner.

### SUMMARY OF THE INVENTION

Certain embodiments of the invention provide vertebroplasty systems and methods for sensing retrograde bone cement flows that can migrate along a fractured path toward a pedicle and risk leakage into the spinal canal. The physician can be alerted instantaneously of cement migration in a direction that may impinge on nerves or the spinal cord. Other embodiments include integrated sensing systems and energy delivery systems for applying energy to tissue and/or to bone cement that migrates in a retrograde direction wherein the energy polymerizes the cement and/or coagulates tissue to create a dam to prevent further cement migration. In another embodiment, the systems provide a cooling system for cooling bone cement in a remote container or injection cannula for controlling and extending the working time of a bone cement. In another embodiment, the bone cement injection system includes a thermal energy emitter for warming bone cement within an injector or for applying sufficient energy to accelerate polymerization and thereby increase the viscosity of the bone cement.

In one embodiment, a computer controller is provided to controls cement inflow parameters from a hydraulic source, the sensing system and energy delivery parameters for selectively heating tissue or polymerizing cement at both the interior and exterior of the injector to thereby control all parameters of cement injection to reduce workload on the physician.

In another embodiment, a lubricous surface layer is provided in the flow passageway of the bone cement injector to inhibit sticking of the bone cement to the wall of the flow channel in the introducer, particularly when heating the cement.

In accordance with one embodiment, a method of treating an abnormal vertebra is provided. The method comprises advancing an elongated member transpedicularly into vertebral cancellous bone, a distal end of the elongated member having an angled surface relative a longitudinal axis of the elongated member, at least a distal flex portion of the elongated member being deflectable away from the longitudinal axis, and creating a curved path in cancellous bone by deflecting at least the distal flex portion of the elongated member via the engagement of said angled surface with bone as the elongated member is advanced into cancellous bone.

In accordance with another embodiment, a bone treatment device is provided. The device comprises an elongated shaft member extending along a longitudinal axis and configured for insertion into cancellous bone. The shaft has a working end comprising a proximal semi-rigid shaft portion, a medial flexible shaft portion and a distal end having a surface that is angled relative to said axis, wherein at least a portion of the working end of the elongated shaft member is configured to deflect away from the longitudinal axis.

In accordance with still another embodiment, a bone treatment device is provided. The device comprises an elongated shaft member extending along a longitudinal axis and configured for insertion into cancellous bone. The shaft has a working end comprising a proximal shaft portion, a medial flexible shaft portion and a distal end having a surface that is angled relative to said axis. The medial flexible shaft portion comprises at least one slideable element actuatable to deflect the distal end of the elongated shaft member away from the longitudinal axis.

In accordance with yet another embodiment, a system for treating an abnormal vertebra is provided. The system comprises an elongated trocar configured for pedicular insertion into vertebral cancellous bone so as to create a curved path in cancellous bone. A distal end of the elongated trocar has an angled surface relative a longitudinal axis of the elongated trocar. At least a distal flex portion of the elongated trocar is configured to deflect away from the longitudinal axis. The system also comprises an elongated injector configured for insertion into the cancellous bone to deliver a bone fill material into the curved path, and a thermal energy emitter disposed in the elongated injector and configured to apply energy to the bone fill material prior to delivery of bone fill material into the curved path in cancellous bone.

These and other objects of the present invention will become readily apparent upon further review of the following drawings and specification.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1 is a schematic perspective view of a hydraulic bone cement injection system and sensing system in accordance with one embodiment.

FIG. 2 is another schematic view of the bone cement injector of FIG. 1.

FIG. 3A is a schematic cross-sectional view of a vertebra showing a first step in one embodiment of a bone cement injection method.

FIG. 3B is a schematic cross-sectional view of the vertebra of FIG. 3A showing a subsequent step in the bone cement injection method.

FIG. 3C is a schematic cross-sectional view similar to FIGS. 3A-3B showing a subsequent step in the bone cement injection method wherein a retrograde flow is detected.

FIG. 4 is a schematic cut-away view of another embodiment of a bone cement injector similar to that of FIGS. 1-2.

FIG. 5 is a schematic cross-sectional view of a distal portion of the bone cement injector of FIGS. 1-2 with a thermal energy emitter in an interior bore of the injector, a sensor system and scratch-resistant insulative exterior coating.

FIG. 6A is a schematic plan view of the working end of a trocar adapted for deflection and for providing a curved path in cancellous bone.

FIG. 6B is a schematic perspective view of the trocar of FIG. 6A together with a cannula (in phantom view).

FIG. 7A is a schematic view of a step of one embodiment of a method of advancing the trocar of FIG. 6A through cortical bone of the pedicle and into cancellous bone.

FIG. 7B is a schematic view of a subsequent step of advancing the trocar into cancellous bone.

FIG. 8 is a schematic view of an alternative embodiment of a trocar.

FIG. 9 is a schematic view of an alternative embodiment of a trocar with an actuatable working end.

### DETAILED DESCRIPTION OF THE PREFERRED EMBODIMENT

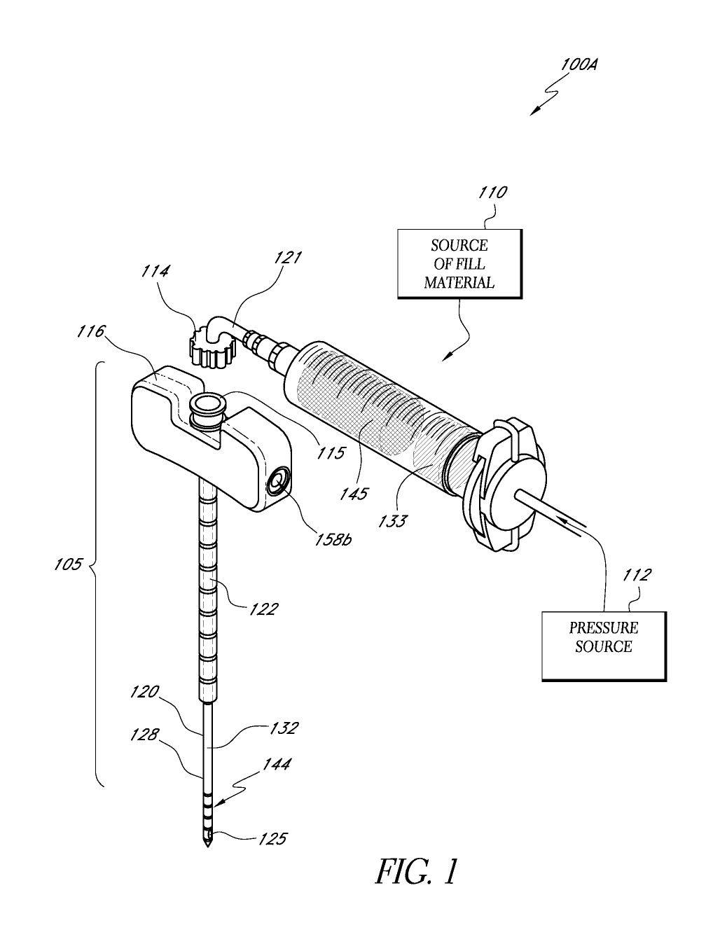

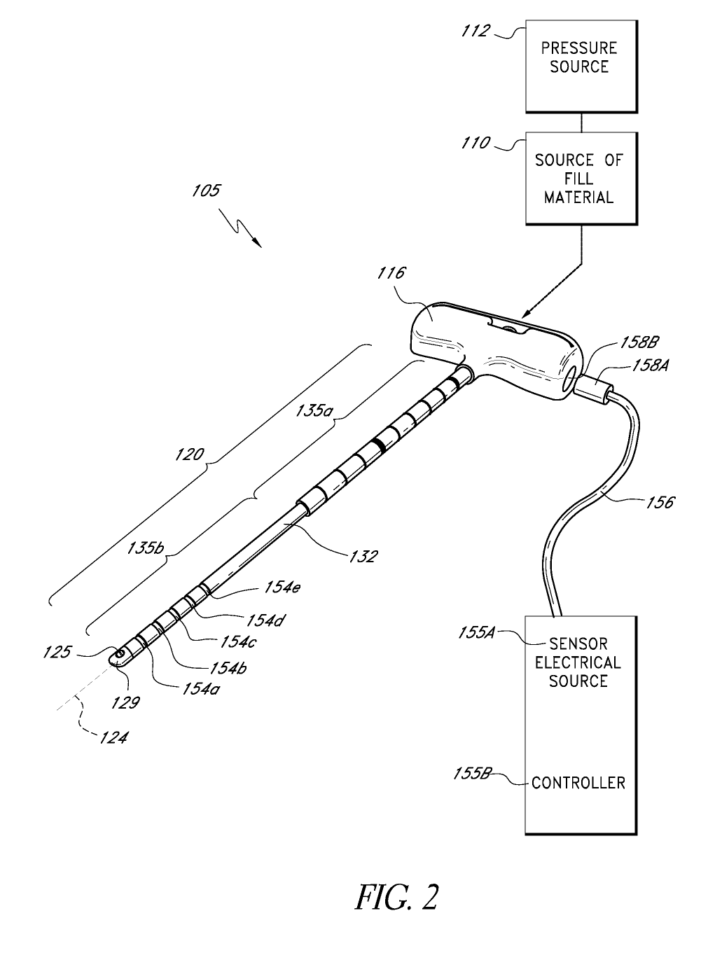

For the purposes of understanding the principles of the invention, reference will now be made to the embodiments illustrated in the drawings and accompanying text that describe the invention. Referring to FIGS. 1-2, one embodiment of a bone fill introducer or injector system 100A is shown that can be used for treatment of the spine in a vertebroplasty procedure. The system 100A includes a bone cement injector 105 that is coupled to source 110 of a bone fill material, wherein the injection of the fill material is carried out by a pressure mechanism or source 112 operatively coupled to the source 110 of bone fill material. In one embodiment, as in FIG. 1, the pressure source 112 can be a hydraulic actuator that can be computer controlled, but the scope of the invention includes a manually operated syringe loaded with bone fill material, or any other pressurized source of fill material. The source 110 of fill material includes a coupling or fitting 114 for sealably locking to a cooperating fitting 115 at a proximal end or handle 116 of the bone cement injector 105 that has an elongated introducer sleeve indicated at 120. In one embodiment, a syringe-type source 110 can be coupled directly to fitting 115 with a flexible, rigid or bendable (deformable) hydraulic tube 121 that extends to the pressure source 112. The fill material then can flow through handle 116 and into a passageway 122 in introducer sleeve 120.

As background, a vertebroplasty procedure using the embodiments disclosed herein can include insertion of the introducer of FIG. 1 through a pedicle of a vertebra for accessing the osteoporotic cancellous bone. The initial aspects of the procedure are similar to a conventional percutaneous vertebroplasty wherein the patient is placed in a prone position on an operating table. The patient is typically under conscious sedation, although general anesthesia is an alternative. The physician injects a local anesthetic (e.g., 1% Lidocaine) into the region overlying the targeted pedicle or pedicles as well as the periosteum of the pedicle(s). Thereafter, the physician uses a scalpel to make a 1 to 5 mm skin incision over each targeted pedicle. Thereafter, the introducer is advanced through the pedicle into the anterior region of the vertebral body, which typically is the region of greatest compression and fracture. The physician confirms the introducer path posterior to the pedicle, through the pedicle and within the vertebral body by anteroposterior and lateral X-Ray projection fluoroscopic views. The introduction of infill material as described below can be imaged several times, or continuously, during the treatment depending on the imaging method.

### Definitions

“Bone fill, fill material, or infill material or composition” includes its ordinary meaning and is defined as any material for infilling a bone that includes an in-situ hardenable material or that can be infused with a hardenable material. The fill material also can include other “fillers” such as filaments, microspheres, powders, granular elements, flakes, chips, tubules and the like, autograft or allograft materials, as well as other chemicals, pharmacological agents or other bioactive agents.

“Flowable material” includes its ordinary meaning and is defined as a material continuum that is unable to withstand a static shear stress and responds with an irrecoverable flow (a fluid)—unlike an elastic material or elastomer that responds to shear stress with a recoverable deformation. Flowable material includes fill material or composites that include a fluid (first) component and an elastic or inelastic material (second) component that responds to stress with a flow, no matter the proportions of the first and second component, and wherein the above shear test does not apply to the second component alone.

“Substantially” or “substantial” mean largely but not entirely. For example, substantially may mean about 10% to about 99.999%, about 25% to about 99.999% or about 50% to about 99.999%.

“Osteoplasty” includes its ordinary meaning and means any procedure wherein fill material is delivered into the interior of a bone.

“Vertebroplasty” includes its ordinary meaning and means any procedure wherein fill material is delivered into the interior of a vertebra.

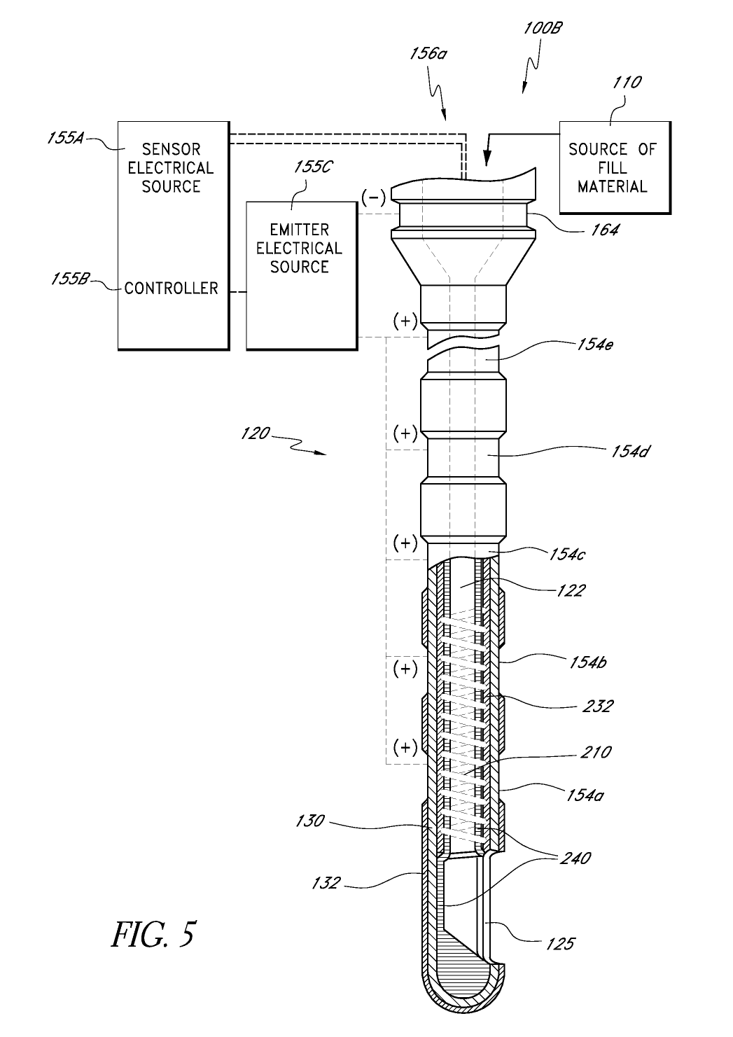

FIGS. 1-5 show that the elongated introducer sleeve 120 of bone cement injector 105 with the interior channel or passageway 122 extends about axis 124 wherein the channel 122 terminates in a distal outlet opening 125. The outlet opening 125 can be a single opening or a plurality of openings disposed about the radially outward surface 128 of the sleeve 120 or an opening at the distal tip 129 the sleeve. The distal tip 129 can be blunt or sharp. In one embodiment, a core portion 130 (see FIG. 5) of sleeve 120 is an electrically conductive metal sleeve, such as a stainless steel hypo tube. The core sleeve portion 130 can have both an exterior insulative coating 132 and an interior insulative coating that will be described in greater detail below.

In one embodiment as shown in FIGS. 1-2, the bone fill system 100A has a container or fill material source 110 that is pressurized by a hydraulic source 112 acting on a floating piston 133 (phantom view) in the container 110, which can be syringe-like. The introducer sleeve 120 can have a proximal portion 135a that is larger in cross-section than a distal portion 135b, and can have corresponding larger and smaller interior channel portions (e.g., passageway 122) therein. This allows for lesser injection pressures to be used since the cement flow needs to travel less distance through the smallest diameter distal portion of the introducer sleeve 120. The distal portion 135b of the introducer 120 can have a cross-section ranging between about 2 mm and 4 mm with a length ranging between about 40 mm and 60 mm. The proximal portion 135a of introducer sleeve 120 can have a cross-section ranging between about 5 mm and 15 mm, or between about 6 mm and 12 mm.

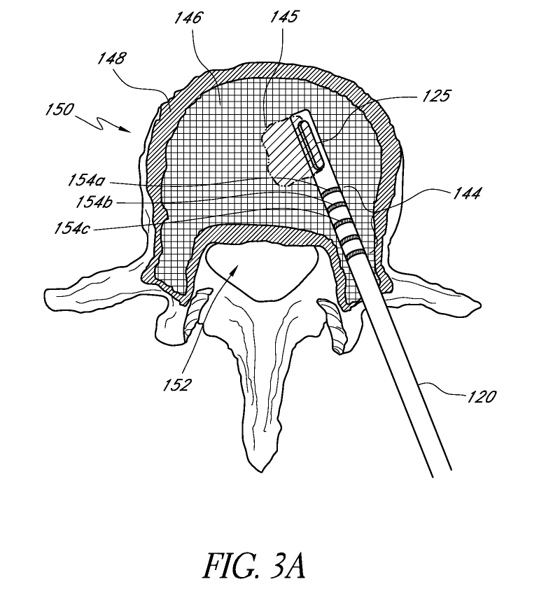

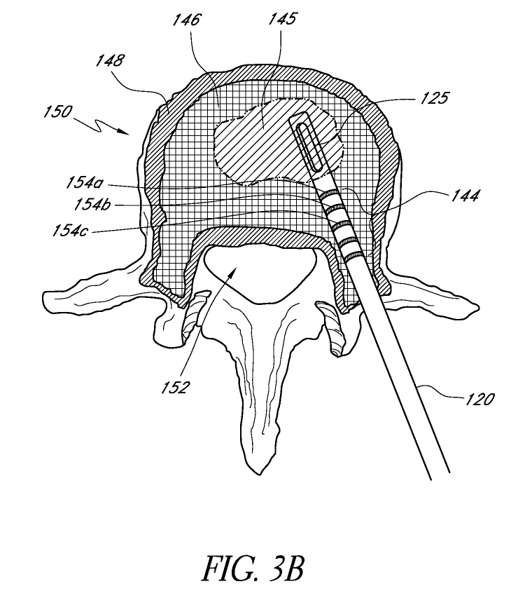

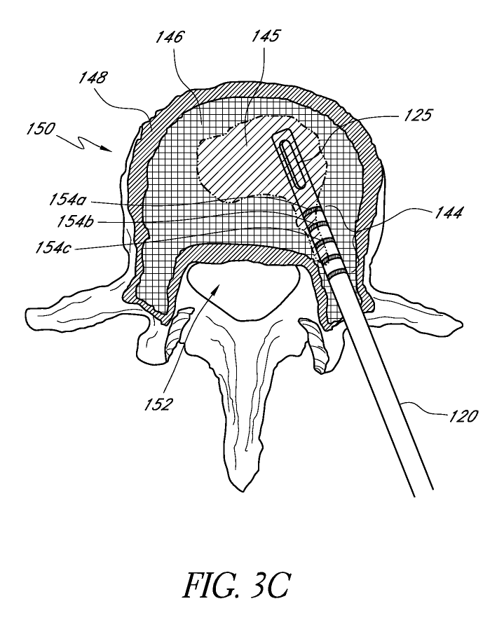

As can be seen in FIGS. 1-2, the exterior surface of introducer sleeve 120 can carry a sensor system 144 that can sense the flow or movement of a fill material or cement 145 (see FIGS. 3A-3C) proximate to the sensors 154a-c of the sensor system 144. The introducer sleeve 120 with such a sensor system 144 is particularly useful in monitoring and preventing extravasation of fill material 145 in a vertebroplasty procedure.

In one embodiment and method of use, referring to FIGS. 3A-3C, the introducer sleeve 120 is used in a conventional vertebroplasty with a single pedicular access or a bi-pedicular access. The fill material 145 can be a bone cement such as PMMA that is injected into cancellous bone 146 which is within the interior of the cortical bone surface 148 of a vertebra 150.

FIGS. 3A-3B show a progressive flow of cement 145 is provided from outlet 125 of introducer sleeve 120 into the interior of the vertebra. FIG. 3A illustrates an initial flow volume with FIG. 3B illustrating an increased flow volume of cement 145. FIG. 3C depicts a situation that is known to occur where bone is fractured along the entry path of the introducer 120 and wherein the cement 145 under high injection pressures finds the path of least resistance to be at least partly in a retrograde direction along the surface of introducer 120. The retrograde flow of cement 145 as in FIG. 3C, if allowed to continue, could lead to cement extravasation into the spinal canal 152, which can lead to serious complications. As can be understood from FIG. 3C, the sensor system 144 can be actuated when cement 145 comes into contact with, or proximate to, the sensors 154a-c of the sensor system 144. In one embodiment shown in FIGS. 2-3C, the sensor system 144 comprises a plurality of spaced apart exposed electrodes or electrode portions (e.g., electrodes 154a, 154b, 154c etc.) that operate as the sensors 154a-e. Though the illustrated embodiments show that the sensor system 144 includes up to five sensors 154a-e, one of ordinary skill in the art will recognize that the sensor system 144 can include more or fewer sensors. The sensors 154a-c are coupled to a sensor electrical source 155A via a cable 156 and a plug 158a connected to electrical connector 158b in the proximal handle end 116 of the introducer 120, wherein the electrical source 155A can carry a low voltage direct current or Rf current between the opposing potentials of spaced apart electrodes 154a-e. The voltage can be from about 0.1 volt to 500 volts, or from about 1 volt to 5 volts and can create a current path through the tissue between a pair of electrodes 154a-e. The current can be continuous, intermittent and/or multiplexed between different electrode pairs or groups of electrodes 154a-e. The arrangement of electrodes 154a-e can be spaced apart ring-type electrodes and axially spaced apart as shown in FIGS. 1 and 2. In another embodiment, the electrodes can be discrete elements, helically spaced electrodes, or can be miniaturized electrodes as in thermocouples, MEMS devices or any combination thereof The number of sensors or electrodes 154 can range from about 1 to 100 and can cooperate, in one embodiment, with a ground pad or other surface portion of the sleeve 120. In one embodiment, the electrodes 154 can include a PTC or NTC material (positive temperature coefficient of resistance or negative temperature coefficient of resistance) to thereby function as a thermistor to allow the measurement of temperature, as well as functioning as a sensor. The sensor system 144 can include a controller 155B (FIG. 2) that measures at least one selected parameter of the current flow to determine a change in a parameter (e.g., impedance). When the bone cement 145, which in one embodiment is non-conductive, contacts one or more electrodes 154a-e of the sensor system 144, the controller 155B identifies a change in the selected electrical parameter and generates a signal to the operator. The scope of the invention includes sensor systems capable of sensing a change in electrical properties, reflectance, fluorescence, magnetic properties, chemical properties, mechanical properties or a combination thereof.

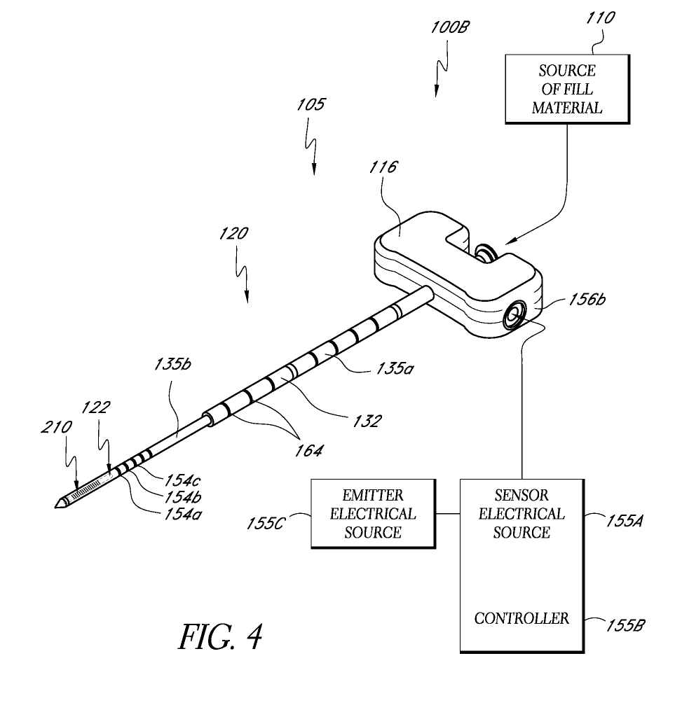

Now referring to FIGS. 4 and 5, an alternative system 100B includes a bone cement injector 105 that is similar to the injector 105 of FIGS. 1-2, but with a different embodiment of a sensor system together with an additional electrical energy delivery system for applying energy to the fill material 145 for altering its viscosity. In this embodiment, the ring electrode portions (i.e. electrodes 154a, 154b, 154c, etc. in phantom view) are exposed portions of the metal core portion 130 of the sleeve 120 (see FIG. 5) that is coupled via lead 156a to electrical source 155A. The electrode portions 154a, 154b, 154c can have a first polarity (+) that cooperates with one or more second polarity (−) return electrodes 164 in a more proximal portion of the sleeve 120 coupled by lead 156b to the sensor electrical source 155A. In this embodiment, current flows through the multiple electrode portions 154a, 154b, 154c, etc. and then through engaged tissue to the return electrodes 164, wherein the current flow can provide a signal of certain parameters (e.g., impedance) before and during an initial injection of cement 145, as in FIGS. 3A-3B. When there is a retrograde flow of cement 145, as in FIG. 3C, that covers one or more electrode portions 154a, 154b, 154c, etc. then the electrical parameter (e.g., impedance) changes to thus signal the operator that such a retrograde flow has contacted or covered an electrode portion 154a, 154b, 154c, etc. The change in parameter can be a rate of change in impedance, a change in impedance compared to a data library, etc. which will signal the operator of such a retrograde flow. In response to such a signal, the controller 155B also can in one embodiment automatically terminate the activation of the pressure source 112.

In the system of FIGS. 4 and 5, the bone fill injection system 100B further includes a thermal energy emitter 210 within the interior channel 122 of the introducer 120 (e.g., in the distal section of the introducer 120) for heating a flow of bone cement 145. In one embodiment, the thermal energy emitter 210 is a resistive heating element 210 that can elevate the temperature of cement 145 to at least 50° C., at least 60° C., at least 70° C. or at least 80° C. The resistive element 210 can be coupled to an emitter electrical source 155C, as depicted in FIGS. 4 and 5, together with controller 155B. In one embodiment, the controller 155B can control cement inflow parameters such as variable flow rates, constant flow rates and/or pulsed flows, as well as control the delivery of energy to the bone fill material 145 via the thermal energy emitter 210. The thermal energy delivery can accelerate polymerization and increase the viscosity of a PMMA or similar bone cement, as disclosed in the co-pending U.S. Patent Applications listed below. In another embodiment, the thermal energy emitter also can be an Rf emitter adapted for ohmically heating a bone cement that carries electrically conductive compositions, as disclosed in the below co-pending U.S. patent application Ser. No. 11/165,652 filed Jun. 24, 2005; Ser. No. 11/165,651 filed Jun. 24, 2005; Ser. No. 11/208,448 filed Aug. 20, 2005; and Ser. No. 11/209,035 filed Aug. 22, 2005. In another embodiment, the thermal energy emitter 210 can deliver thermal energy to bone cement and can be selected from the group consisting of a resistively heated emitter, a light energy emitter, an inductive heating emitter, an ultrasound source, a microwave emitter and any other electromagnetic energy emitter. In the embodiment of FIGS. 4 and 5, the controller 155B can control all parameters of (i) heating the bone cement, (ii) the cement injection pressure and/or flow rate, (iii) energy delivery to cement flows in or proximate the distal end of the introducer and (iv) energy delivery to sense retrograde flows about the exterior surface of the introducer.

In one embodiment depicted in FIG. 5, the resistive heating element 210 comprises a helically wound coil of a resistive material in the interior bore or passageway 122 of the introducer 120. The heating element 210 optionally can be further formed from, or coated with, a positive temperature coefficient material and coupled to a suitable voltage source to provide a constant temperature heater, as is known in the art. As can be seen in FIG. 5, the heating element 210 can be disposed within an insulative coating 232 in the interior of the core sleeve 130, which can be a conductive metal as described above.

With continued reference to the embodiment in FIG. 5, the exterior surface of sleeve 120 can have an insulative, scratch-resistant coating 132 that can comprises a thin layer of an insulative amorphous diamond-like carbon (DLC) or a diamond-like nanocomposite (DCN). It has been found that such coatings have high scratch resistance, as well as lubricious and non-stick characteristics that are useful in bone cement injectors of the invention. Such coatings are particularly useful for an introducer sleeve 120 that can carry electrical current for (i) impedance sensing purposes; (ii) for energy delivery to bone fill material 145; and/or (iii) ohmic heating of tissue. For example, when inserting a bone cement injector 105 through the cortical bone surface 148 of a pedicle and then into the interior of a vertebra 150, it is important that the exterior insulative coating portions 132 do not fracture, chip or scratch to thereby ensure that the electrical current carrying functions of the injector 105 are not compromised.

The amorphous diamond-like carbon coatings and the diamond-like nanocomposites are available from Bekaert Progressive Composites Corporations, 2455 Ash Street, Vista, Calif. 92081 or its parent company or affiliates. Further information on the coating can be found at: http://www.bekaert.com/bac/Products/Diamond-like%20coatings.htm, the contents of which are incorporated herein by reference. The diamond-like coatings comprise amorphous carbon-based coatings with high hardness and low coefficient of friction. The amorphous carbon coatings exhibit non-stick characteristics and excellent wear resistance. The coatings can be thin, chemically inert and have a very low surface roughness. In one embodiment, the coatings have a thickness ranging between 0.001 mm and 0.010 mm; or between 0.002 mm and 0.005 mm. The diamond-like carbon coatings are a composite of sp2 and sp3 bonded carbon atoms with a hydrogen concentration between 0 and 80%. Another diamond-like nanocomposite coatings (a-C:H/a-Si:O; DLN) is made by Bakaert and is suitable for use in the bone cement injector of the invention. The materials and coatings are known by the names Dylyn®Plus, Dylyn®/DLC and Cavidur®.

FIG. 5 further illustrates another aspect of bone cement injector 105 that again relates to the thermal energy emitter (resistive heater 210) within interior passageway 122 of introducer 120. In one embodiment, it has been found that it is advantageous to provide a lubricious surface layer 240 within the interior of resistive heater 210 to ensure uninterrupted cements flows through the thermal emitter 210 without sticking to the passageway 122. In one embodiment, surface layer 240 is a fluorinated polymer, such as Teflon® or polytetrafluroethylene (PTFE). Other suitable fluoropolymer resins can be used such as FEP and PFA. Other materials also can be used such as FEP (Fluorinated ethylenepropylene), ECTFE (Ethylenechlorotrifluoroethylene), ETFE, Polyethylene, Polyamide, PVDF, Polyvinyl chloride and silicone. The scope of the invention includes providing a bone cement injector having a flow channel extending therethrough with at least one open termination 125, wherein a surface layer 240 within the flow channel has a static coefficient of friction of less than 0.5, less than 0.2, or less than 0.1.

In another embodiment, the bone cement injector 105 has a flow channel 122 extending therethrough with at least one open termination 125, wherein at least a portion of the surface layer 240 of the flow channel is ultrahydrophobic or hydrophobic which may better prevent a hydrophilic cement from sticking.

In another embodiment, the bone cement injector has a flow channel 122 extending therethrough with at least one open termination 125, wherein at least a portion of the surface layer 240 of the flow channel is hydrophilic for which may prevent a hydrophobic cement from sticking.

In another embodiment, the bone cement injector has a flow channel 122 extending therethrough with at least one open termination in a distal end thereof, wherein the surface layer 240 of the flow channel has high dielectric strength, a low dissipation factor, and/or a high surface resistivity.

In another embodiment, the bone cement injector has a flow channel 122 extending therethrough with at least one open termination 125 in a distal end thereof, wherein the surface layer 240 of the flow channel is oleophobic. In another embodiment, the bone cement injector has a flow channel 122 extending therethrough with at least one open termination 125 in a distal end thereof, wherein the surface layer 240 of the flow channel has a substantially low coefficient of friction polymer or ceramic.

In another embodiment, the bone cement injector has a flow channel 122 extending therethrough with at least one open termination 125 in a distal end thereof, wherein the surface layer 240 of the flow channel has a wetting contact angle greater than 70°, greater than 85°, and greater than 100°.

In another embodiment, the bone cement injector has a flow channel 122 extending therethrough with at least one open termination in a distal end thereof, wherein the surface layer 240 of the flow channel has an adhesive energy of less than 100 dynes/cm, less than 75 dynes/cm, and less than 50 dynes/cm.

The apparatus above also can be configured with any other form of thermal energy emitter that includes the non-stick and/or lubricious surface layer as described above. In one embodiment, the thermal energy emitter can comprise at least in part an electrically conductive polymeric layer. In one such embodiment, the electrically conductive polymeric layer has a positive temperature coefficient of resistance.

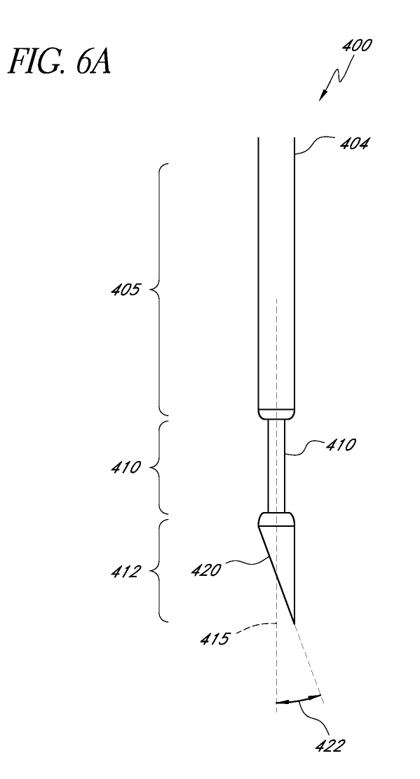

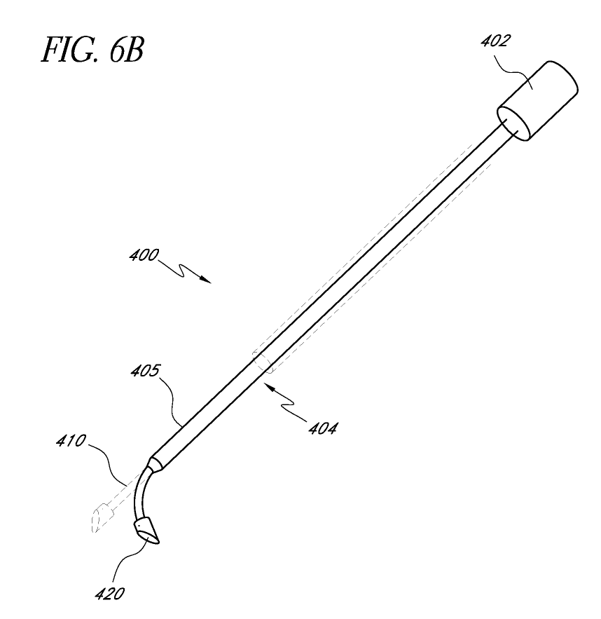

FIG. 6A is a plan view of the working end of an elongated trocar or treatment device 400 that can be used for penetrating into the cancellous bone 146 of the vertebra 150 and creating a curved path in such bone in a particular plane. The trocar 400 can have a proximal handle 402 (FIG. 6B) and include an elongated shaft 404 wherein the working end that penetrates bone has a proximal shaft portion 405 that optionally is slightly flexible or substantially rigid. The working end extends distally and includes medial shaft portion 410 that transitions to distal tip portion 412. The trocar 400 can be made of any suitable material used for spinal surgical procedures. As can be seen in FIG. 6A-6B, the medial shaft portion 410 can be made of a more flexible material, such as a superelastic alloy, and in one embodiment has a reduced diameter cross-section relative to the more proximal shaft 405 and the tip portion 412. The more flexible medial shaft portion 410 allows the shaft to flex or deflect relative to an axis 415 that extends generally along the elongated shaft 404. The tip portion 412 can have an angled face or surface 420 that when introduced through cancellous bone 146, causes at least the tip 412 and medial portion 410 to deflect away from the axis 415 to create a curved path in the cancellous bone 146. The angle 422 of the surface 420 relative to axis 415 can range from about 10° to 75°, or 20° to 50°.

The axial length of the flexible medial shaft portion 410 can range from 1 mm. to 20 mm, or from 2 mm. to 15 mm, or from 4 mm. to 10 mm. In one embodiment, the flexible medial shaft portion 410, as shown in FIGS. 6A-6B, has a single wire-like element. However, the medial shaft portion also can comprise a plurality of wire-like elements. The flexible medial shaft portion 410 can be on-axis, off-axis, axis-symmetric or non-symmetric relative to the axis 415.

In one embodiment as in FIGS. 6A-6B, the flexible medial shaft portion 410 has a reduced cross-section relative to the shaft 404. However, in another embodiment, the medial shaft portion 410 can have a cross-section that matches the shaft 404 and/or tip 412, or can be a helical spring-like element (not shown). The medial portion 410 also can have a flexible polymer jacket that has a cross-section similar to the shaft 402 (not shown). In FIG. 6B, the trocar 400 is shown with a cannula 425 in phantom view. The cannula 425 can extend into the cancellous bone 146 and be advanced or retracted to function as a constraining sleeve about a portion of the shaft to maintain said shaft portion in a linear configuration.

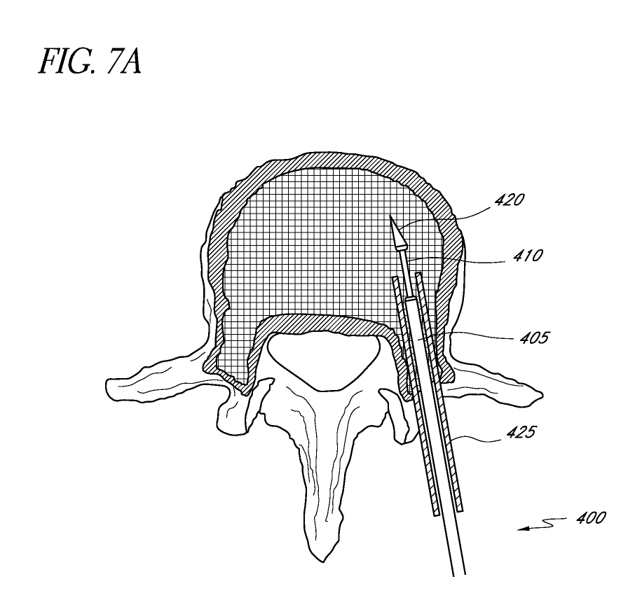

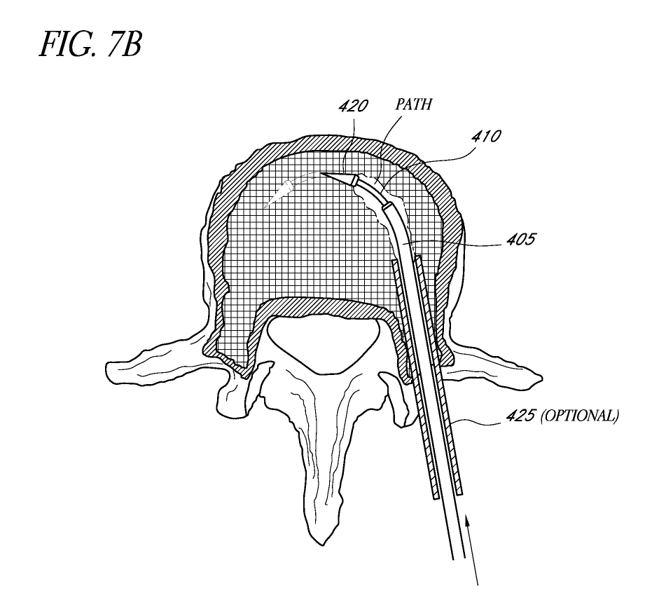

FIGS. 7A-7B illustrate a method for treating an abnormal vertebra by advancing an elongated shaft member 404 transpedicularly or parapedicularly into vertebral cancellous bone 146, wherein a distal end 412 of the shaft member 404 has an angled surface 420 relative a longitudinal axis of the shaft, and wherein said angled surface 420 engages bone which causes deflection forces to deflect a distal flex region 410 of the shaft; and wherein further advancing the shaft 404 with the deflected distal flex region 410 creates a curved path in said cancellous bone. Subsequently, the method includes introducing a bone fill material injector, such as injector 105 in FIGS. 1-5, into the curved path and injecting bone fill material 145 therefrom.

The method can include introducing the fill material injector 105 over the elongated proximal and medial shaft portion (405 and 410) and into the curved path. In another embodiment, the fill material injector 105 can be introduced into the curved path after withdrawal of the elongated shaft portions.

The method can further include applying thermal energy to the fill material 145 (e.g., via the energy emitter 210) in the injector 105, as described in earlier embodiments. The application of thermal energy can be provided from at least one of an electrical source, a resistive heat source, a light source, a microwave source, and inductive heating source, an Rf source, an ultrasound source and a source of heated vapor. The bone fill material 145 can be an exothermic bone cement, such as PMMA. In one embodiment, the use of vapor injection is used to emulsify the bone fill material.

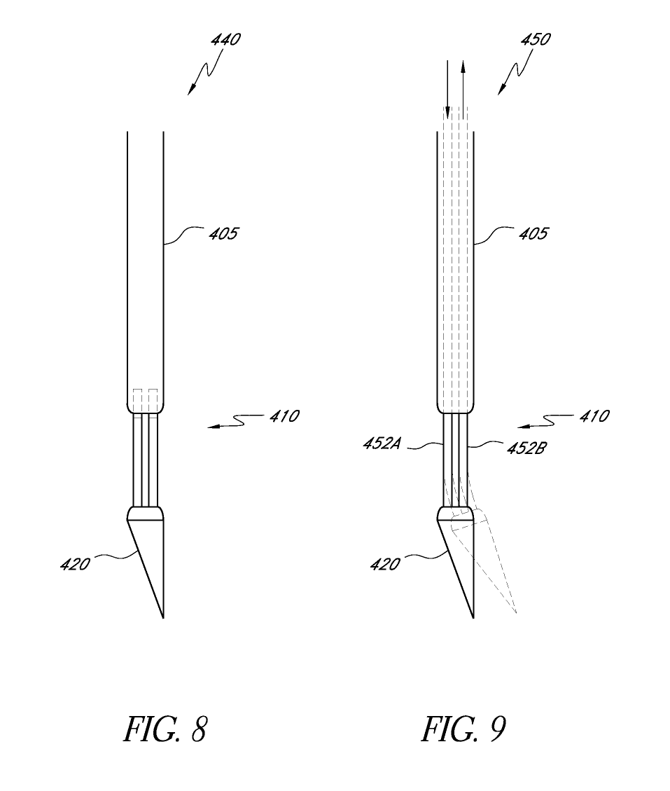

In another embodiment shown in FIG. 8, the working end of trocar 440 includes a flexible medial portion 410 with multiple fixed elements. FIG. 9 illustrates another trocar 450 that includes actuatable, slideable elements 452A and 452B that can be moved axially to deflect the surface 420 of the tip and the curvature of medial shaft portion 410 to control the arc of the curved path formed with the trocar 450.

In another apparatus and method, the introducing step includes an actuating step wherein energy is applied to tissue from the distal end 412 of the trocar to the body structure. The energy-applying step can include applying energy selected from the group of thermal energy, ultrasound energy, vibration energy, mechanical energy, light energy, electromagnetic energy, radiofrequency energy, microwave energy and chemical energy. The effect of such energy delivery is for cutting tissue, coagulating tissue, sealing tissue, damaging tissue and vaporizing tissue.

In another method, a trocar shaft and tip are advanced to create an arc in cancellous bone 146 in a vertebra 150, wherein the working end extends at least about 90° in the arc configuration in the cancellous bone 146. The method further includes causing the working end to extend in an arc of at least about 120°, 150°, 180°, 210° and 240°.

In another method, two complementary trocars 400 each with a working end can be introduced into the vertebra 150, one from each pedicle or from opposite parapedicular location.

In another embodiment and method, a flexible or shape memory bone cement injector working end (not shown) can be introduced into the path created by trocar 400, and then cement can be injected from a plurality of ports along the length of the injector working end, wherein the ports are oriented in a selected direction toward the center of the vertebra. The working end of the injector can have the heating element 210, as described above, or preferably a polymeric PTCR heating element. In such an embodiment, the step of applying thermal energy is accomplished by a resistive heating element that comprises a sleeve fabricated of a positive temperature coefficient of resistance (PTCR) material.

In another embodiment, the step applying thermal energy is accomplished by light energy from an LED, or from at least one of coherent light and non-coherent light.

In another embodiment, the step of applying thermal energy includes the heat of vaporization from a vapor, which can be introduced through a channel in the injector to interact with the cement. Such a vapor can be generated from water, saline or any other biocompatible fluid.

In related methods, the system can use any suitable energy source, other that radiofrequency energy, to accomplish the purpose of altering the viscosity of the fill material 145. The method of altering fill material can be at least one of a radiofrequency source, a laser source, a microwave source, a magnetic source and an ultrasound source. Each of these energy sources can be configured to preferentially deliver energy to a cooperating, energy sensitive filler component carried by the fill material 145. For example, such filler can be suitable chromophores for cooperating with a light source, ferromagnetic materials for cooperating with magnetic inductive heating means, or fluids that thermally respond to microwave energy.

The scope of the invention includes using additional filler materials such as porous scaffold elements and materials for allowing or accelerating bone ingrowth. In any embodiment, the filler material can comprise reticulated or porous elements of the types disclosed in co-pending U.S. patent application Ser. No. 11/146,891, filed Jun. 7, 2005, titled “Implants and Methods for Treating Bone” which is incorporated herein by reference in its entirety and should be considered a part of this specification. Such fillers also can carry bioactive agents. Additional fillers, or the conductive filler, also can include thermally insulative solid or hollow microspheres of a glass or other material for reducing heat transfer to bone from the exothermic reaction in a typical bone cement component.

The above description of the invention is intended to be illustrative and not exhaustive. Particular characteristics, features, dimensions and the like that are presented in dependent claims can be combined and fall within the scope of the invention. The invention also encompasses embodiments as if dependent claims were alternatively written in a multiple dependent claim format with reference to other independent claims. Specific characteristics and features of the invention and its method are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

Of course, the foregoing description is that of certain features, aspects and advantages of the present invention, to which various changes and modifications can be made without departing from the spirit and scope of the present invention. Moreover, the bone treatment systems and methods need not feature all of the objects, advantages, features and aspects discussed above. Thus, for example, those skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed bone treatment systems and methods.

What is claimed is:

1. A method of treating an abnormal vertebra, comprising:
advancing an elongated member transpedicularly into vertebral cancellous bone, a distal end of the elongated member having an angled surface relative a longitudinal axis of the elongated member, at least a distal flex portion of the elongated member being deflectable away from the longitudinal axis; and
creating a curved path in cancellous bone by deflecting at least the distal flex portion of the elongated member via the engagement of said angled surface with bone as the elongated member is advanced into cancellous bone.

2. The method of claim 1, further comprising introducing a bone fill material injector into the vertebra so that the injector is in communication with the curved path and injecting a bone fill material through the injector into the curved path.

3. The method of claim 2, wherein the bone fill material injector is introduced into the vertebra over the elongated member.

4. The method of claim 2, wherein the bone fill material injector is introduced into the curved path after withdrawal of the elongated member from the curved path.

5. The method of claim 2, further comprising applying thermal energy to the bone fill material from an emitter in the injector.

6. The method of claim 5, wherein the application of thermal energy is provided by at least one of an electrical source, a resistive heat source, a light source, a microwave source, and inductive heating source, an Rf source and an ultrasound source.

7. The method of claim 5, wherein the bone fill material is an exothermic bone cement.

8. A bone treatment device, comprising an elongated shaft member extending along a longitudinal axis and configured for insertion into cancellous bone, the shaft having a working end comprising a proximal semi-rigid shaft portion, a medial flexible shaft portion and a distal end having a surface that is angled relative to said axis, wherein at least a portion of the working end of the elongated shaft member is configured to deflect away from the longitudinal axis.

9. The bone treatment device of claim 8, wherein the medial flexible shaft portion has a smaller cross-sectional dimension than the proximal rigid shaft portion.

10. The bone treatment device of claim 8, wherein the medial flexible shaft portion comprises a superelastic material.

11. The bone treatment device of claim 8, wherein the medial flexible shaft portion is off-axis.

12. The bone treatment device of claim 8, wherein the medial flexible shaft portion is non-symmetrical relative to said axis.

13. The bone treatment device of claim 8, wherein the medial flexible shaft portion comprises a single wire-like element.

14. The bone treatment device of claim 8, wherein the medial flexible shaft portion includes at least one wire-like element and a flexible polymer jacket.

15. The bone treatment device of claim 8, wherein the distal end surface is angled between about 10° and 75° relative to said axis.

16. The bone treatment device of claim 8, wherein the distal end surface is angled between about 20° and 50° relative to said axis.

17. The bone treatment device of claim 8, wherein the medial flexible shaft portion has an axial length ranging between about 1 mm and 20 mm.

18. The bone treatment device of claim 8 wherein the medial flexible shaft portion has an axial length ranging between about 4 mm. and 10 mm.

19. A bone treatment device, comprising an elongated shaft member extending along a longitudinal axis and configured for insertion into cancellous bone, the shaft having a working end comprising a proximal shaft portion, a medial flexible shaft portion and a distal end having a surface that is angled relative to said axis, wherein the medial flexible shaft portion comprises at least one slideable element actuatable to deflect the distal end of the elongated shaft member away from the longitudinal axis.

20. The bone treatment device of claim 19, wherein the slideable element is actuatable from a proximal handle end of the elongated shaft member.

21. The bone treatment device of claim 19, wherein the medial flexible shaft portion comprises two slideable elements, each of the elements actuatable to move axially relative to the proximal shaft portion to deflect the distal end of the elongated shaft member away from the longitudinal axis

22. A system of treating an abnormal vertebra, comprising:
an elongated trocar configured for pedicular insertion into vertebral cancellous bone so as to create a curved path in cancellous bone, a distal end of the elongated trocar having an angled surface relative a longitudinal axis of the elongated trocar, at least a distal flex portion of the elongated trocar configured to deflect away from the longitudinal axis;
an elongated injector configured for insertion into the cancellous bone to deliver a bone fill material into the curved path; and
a thermal energy emitter disposed in the elongated injector and configured to apply energy to the bone fill material prior to delivery of bone fill material into the curved path in cancellous bone.

23. The system of claim 22, wherein the injector is configured for introduction into the vertebra over the elongated trocar.

24. The system of claim 22, wherein the emitter is coupled to an external energy source.

25. The system of claim 24, wherein the external energy source is at least one of an electrical source, a resistive heat source, a light source, a microwave source, and inductive heating source, an Rf source and an ultrasound source.
